(19) United States
(12) Patent Application Publication
(10) Pub. No.: US 2018/0303795 A1
(43) Pub. Date: Oct. 25, 2018

(54) METHOD OF TREATING ROSACEA

(71) Applicants: Hedayat Adhami, Los Angeles, CA (US); Afshin Shawn Adhami, Los Angeles, CA (US)

(72) Inventors: Hedayat Adhami, Los Angeles, CA (US); Afshin Shawn Adhami, Los Angeles, CA (US)

(21) Appl. No.: 15/962,537

(22) Filed: Apr. 25, 2018

Related U.S. Application Data
(60) Provisional application No. 62/489,980, filed on Apr. 25, 2017.

(51) Int. Cl. A61K 31/375; A61P 17/00; A61K 9/00

(52) U.S. Cl. CPC A61K 31/375; A61K 9/0019; A61P 17/00

(57) ABSTRACT

A method of treating, preventing, and/or managing rosacea in a subject includes the steps of administering intravenously to the subject a therapeutically effective amount of vitamin C. The therapeutically effective amount is at least one gram of vitamin C. The administering may be performed once every day or every other day. Alternatively, the administering may be stopped for a one week in a month period, or for one week in any eight month period. Alternatively, the administering may be performed once every day for a week and off for a week, wherein the method is then repeated or performed once every other day for a week and off for a week, wherein the method is then repeated.

### CROSS-REFERENCE TO RELATED APPLICATIONS

This non-provisional patent application claims priority to provisional application 62/489,980 filed on Apr. 25, 2017, the contents of which are fully incorporated herein with this reference.

### DESCRIPTION

### Field of the Invention

The present invention generally relates to rosacea and treatments thereof. More particularly, the present invention relates to vitamin C intravenously taken to reduce and/or eliminate rosacea.

### Background of the Invention

Rosacea is a long term skin condition characterized by facial redness, small and superficial dilated blood vessels on facial skin, papules, pustules, and swelling. Rosacea typically begins as redness on the central face across the cheeks, nose, or forehead, but can also less commonly affect the neck, chest, ears, and scalp. In some cases, additional signs, such as semipermanent redness, dilation of superficial blood vessels on the face, red domed papules (small bumps) and pustules, red gritty eyes, burning and stinging sensations, and in some advanced cases, a red lobulated nose (rhinophyma), may develop.

Rosacea affects all ages and has four subtypes, three affecting the skin and the fourth affecting the eyes (ocular rosacea). It primarily affects people of northwestern European descent and has been nicknamed the “curse of the Celts” by some in Britain and Ireland, although such a connection has been questioned. Rosacea is almost three times more common in women. It is commonly found in people between the ages of 30 and 50, and is more common in Caucasians.

The exact cause of rosacea is unknown. Triggers that cause episodes of flushing and blushing play a part in its development. Exposure to temperature extremes, strenuous exercise, heat from sunlight, severe sunburn, stress, anxiety, cold wind, and moving to a warm or hot environment from a cold one, such as heated shops and offices during the winter, can each cause the face to become flushed. Certain foods and drinks can also trigger flushing, such as alcohol, foods and beverages containing caffeine (especially hot tea and coffee), foods high in histamines, and spicy foods.

Medications and topical irritants have also been known to trigger rosacea flares. Some acne and wrinkle treatments reported to cause rosacea include microdermabrasion and chemical peels, as well as high dosages of isotretinoin, benzoyl peroxide, and tretinoin. Steroid-induced rosacea is caused by the use of topical or nasal steroids. These steroids are often prescribed for seborrheic dermatitis. Dosage should be slowly decreased and not immediately stopped to avoid a flare-up.

Treating rosacea varies depending on severity and subtypes. A subtype-directed approach to treating rosacea patients is recommended to dermatologists. Mild cases are often not treated at all, or are simply covered up with normal cosmetics. Therapy for the treatment of rosacea is not curative, and is best measured in terms of reduction in the amount of facial redness and inflammatory lesions, a decrease in the number, duration, and intensity of flares, and concomitant symptoms of itching, burning, and tenderness. The two primary modalities of rosacea treatment are topical and oral antibiotic agents. Laser therapy has also been classified as a form of treatment. While medications often produce a temporary remission of redness within a few weeks, the redness typically returns shortly after treatment is suspended. Long-term treatment, usually one to two years, may result in permanent control of the condition for some patients. Lifelong treatment is often necessary, although some cases resolve after a while and go into a permanent remission. Other cases, left untreated, worsen over time.

Avoiding triggers that worsen the condition can help reduce the onset of rosacea, but alone will not normally lead to remission except in mild cases. Keeping a journal is sometimes recommended to help identify and reduce food and beverage triggers. Because sunlight is a common trigger, avoiding excessive exposure to the sun is widely recommended. Some people with rosacea benefit from daily use of a sunscreen; others opt for wearing hats with broad brims. Like sunlight, emotional stress can also trigger rosacea. People who develop infections of the eyelids must practice frequent eyelid hygiene. Managing pretrigger events such as prolonged exposure to cool environments can directly influence warm room flushing.

Medications with good evidence include ivermectin and azelaic acid creams and brimonidine, doxycycline, and isotretinoin by mouth. Lesser evidence supports metronidazole cream and tetracycline by mouth. Metronidazole is thought to act through anti-inflammatory mechanisms, while azelaic acid is thought to decrease cathelicidin production. Oral antibiotics of the tetracycline class such as doxycycline and oxytetracycline are also commonly used and thought to reduce papulopustular lesions through anti-inflammatory actions rather than through their antibacterial capabilities. Using alpha-hydroxy acid peels may help relieve redness caused by irritation, and reduce papules and pustules associated with rosacea. Oral antibiotics may help to relieve symptoms of ocular rosacea. If papules and pustules persist, then sometimes isotretinoin can be prescribed. The flushing and blushing that typically accompanies rosacea is typically treated with the topical application of alpha agonists such as brimonidine and less commonly oxymetazoline or xylometazoline. Treatment has also typically been with doxycycline, tetracycline, or metronidazole. Other treatments with tentative benefit include brimonidine cream, ivermectin cream, and isotretinoin

Dermatological vascular laser (single wavelength) or intense pulsed light (broad spectrum) machines offer one of the treatments for rosacea, in particular the erythema (redness) of the skin. They use light to penetrate the epidermis to target the capillaries in the dermis layer of the skin. The light is absorbed by oxyhemoglobin, which heats up, causing the capillary walls to heat up to 70° C. (158° F.), damaging them, and causing them to be absorbed by the body's natural defense mechanism. With a sufficient number of treatments, this method may even eliminate the redness altogether, though additional periodic treatments will likely be necessary to remove newly formed capillaries.

CO2 lasers can be used to remove excess tissue caused by phymatous rosacea. CO2 lasers emit a wavelength that is absorbed directly by the skin. The laser beam can be focused into a thin beam and used as a scalpel or defocused and used to vaporize tissue. Low-level light therapies have also been used to treat rosacea. Photorejuvenation can also reportedly be used to improve the appearance of rosacea and reduce the redness associated with it.

As can be understood by those skilled in the art, there is no perfect cure for rosacea, as many experts recommend a varying range of treatments. Applying topical medications can become a burden over time. Laser treatments are expensive. Oral medications are not effective for everyone. Accordingly, an improved method of treating rosacea that is safe, easy to administer and effective is always of need. The present invention fulfills these needs and provides other related advantages.

### SUMMARY OF THE INVENTION

A method of treating, preventing, or managing rosacea in a subject includes the steps of administering intravenously to the subject a therapeutically effective amount of vitamin C. The therapeutically effective amount is at least one gram of vitamin C. The administering may be performed once every day or every other day. Alternatively, the administering may be stopped for a one week in a month period, or for one week in any eight month period. Alternatively, the administering may be performed once every day for a week and off for a week, wherein the method is then repeated or performed once every other day for a week and off for a week, wherein the method is then repeated. The vitamin C may be Redoxon®.

Other features and advantages of the present invention will become apparent from the following more detailed description, when taken in conjunction with the accompanying drawings, which illustrate, by way of example, the principles of the invention.

### DETAILED DESCRIPTION OF THE PREFERRED EMBODIMENTS

A method of treating, preventing, or managing rosacea in a subject includes the steps of administering intravenously to the subject a therapeutically effective amount of vitamin C. The therapeutically effective amount is at least one gram of vitamin C. The administering may be performed once every day or every other day. Alternatively, the administering may be stopped for a one week in a month period, or for one week in any eight month period. Alternatively, the administering may be performed once every day for a week and off for a week, wherein the method is then repeated or performed once every other day for a week and off for a week, wherein the method is then repeated.

Vitamin C is also known as ascorbic acid. Ascorbic acid is essential to collagen formation and helps to maintain the integrity of substances of mesenchymal origin, such as blood and blood vessels. The inventors have found if vitamin C is prepared in an injectable formulation, it can be used to effectively treat rosacea.

The vitamin C may be Redoxon. Redoxon® is the name of the first artificially synthesized ascorbic acid, which was vitamin C. Redoxon® was first marketed to the general public in about 1934 which was then the first mass-manufactured synthetic vitamin in history. It is now a brand owned by German pharmaceutical company Bayer and is sold world-wide. The product was developed by a team headed by chemist Tadeusz Reichstein, who discovered a method of synthesizing 30-40 g of vitamin C from 100 g of glucose. This used an intermediate step of creating sorbose using an ingenious bacterial fermentation method discovered by a French researcher, Gabriel Bertrand. In this method, fruit flies were attracted to a mixture of wine, vinegar, yeast bouillon, and sorbitol, a substance easily chemically prepared from glucose. Flies which fed upon sorbitol as a major food substrate excreted bacteria which were able to synthesize sorbose from sorbitol. Using the bacteria, within a few days, it was possible to create 50 grams of sorbose using this method, and it was then easy to synthesize ascorbic acid from this. Despite concern about using the wild strain of bacteria for fermentation-production of sorbose, the process was superior to a rival method of Szent-Györgyi which isolated Vitamin C from capsicum. After sale of the Reichstein process patent to Hoffmann-La Roche, this process became the basis of the corporation's large-scale production of vitamin C. The commercial tablets are compounded from ascorbic acid and sodium bicarbonate. When these are added to water, they react to produce sodium ascorbate, water and carbon dioxide, thus producing a pleasant effervescence. Redoxon® is currently on sale as an oral tablet but contains other additives and minerals. Taking Redoxon orally does not produce the benefits of reducing rosacea. Redoxon® is currently not sold as an injectable formulation.

Although several embodiments have been described in detail for purposes of illustration, various modifications may be made to each without departing from the scope and spirit of the invention. Accordingly, the invention is not to be limited, except as by the appended claims.

What is claimed is:

1. A method of treating, preventing, and/or managing rosacea in a subject, the method comprising the steps of:
administering intravenously to the subject a therapeutically effective amount of vitamin C;
wherein the therapeutically effective amount is at least one gram of vitamin C.

2. The method of claim 1, wherein the administering is performed once every day.

3. The method of claim 2, wherein the administering is stopped for one week in any one month period.

4. The method of claim 2, wherein the administering is stopped for one week in any eight month period.

5. The method of claim 1, wherein the administering is performed once every day for a week and off for a week, wherein the method is then repeated.

6. The method of claim 1, wherein the administering is performed once every other day.

7. The method of claim 1, wherein the administering is performed once every other day for a week and off for a week, wherein the method is then repeated.

8. The method of claim 1, wherein the vitamin C comprises Redoxon®.
